(19) United States
(12) Patent Application Publication
(10) Pub. No.: US 2019/0365744 A1
(43) Pub. Date: Dec. 5, 2019

(54) IMPROVED MULTI TYROSINE KINASE INHIBITOR THERAPY

(71) Applicant: FRAUNHOFER-GESELLSCHAFT ZUR FORDERUNG DER ANGEWANDTEN FORSCHUNG E.V., München (DE)

(72) Inventor: Antje PRASSE, Hannover (DE)

(21) Appl. No.: 16/478,459

(22) PCT Filed: Jan. 16, 2018
(86) PCT No.: PCT/EP2018/050941

§ 371 Date: Jul. 16, 2019

(30) Foreign Application Priority Data
Jan. 16, 2017 (EP) 17151584.4

(51) Int. Cl. A61K 31/496

(52) U.S. Cl. CPC A61K 31/496

(57) ABSTRACT

A pharmaceutical composition or kit has at least one CXCR4 inhibitor and a multi-tyrosine kinase inhibitor. The composition has at least one CXCR4 inhibitor for use in the treatment of cancer or idiopathic pulmonary fibrosis in a subject suffering from the cancer or idiopathic pulmonary fibrosis and receiving a multi-tyrosine kinase inhibitor therapy. A method for identifying whether a subject suffering from cancer or idiopathic pulmonary fibrosis and receiving a multi-tyrosine kinase inhibitor therapy is susceptible to a CXCR4 inhibitor therapy by determining a CXCR4 signal intensity in a PET dataset obtained from the subject, comparing the CXCR signal intensity to a reference, and identifying whether the subject is susceptible based on the results of the comparison.

### PRIORITY AND CROSS REFERENCE TO RELATED APPLICATIONS

This application is the U.S. National Phase Application under 35 U.S.C. § 371 of International Application No. PCT/EP2018/050941, filed Jan. 16, 2018, designating the U.S. and published as WO 2018/130702 A1 on Jul. 19, 2018, which claims the benefit of European Application No. EP 17151584.4, filed Jan. 16, 2017. Any and all applications for which a foreign or a domestic priority is claimed is/are identified in the Application Data Sheet filed herewith and is/are hereby incorporated by reference in their entireties under 37 C.F.R. § 1.57.

### FIELD

The present disclosure is related to CXCR4 inhibitor for use in the treatment of cancer or idiopathic pulmonary fibrosis in a subject suffering from said cancer or idiopathic pulmonary fibrosis, wherein the subject is receiving a multi-tyrosine kinase inhibitor therapy.

### SUMMARY

The present invention relates to the field of therapeutics and diagnostics. In particular, the present invention relates to a pharmaceutical composition or kit comprising (i) at least one CXCR4 inhibitor and (ii) a multi-tyrosine kinase inhibitor. It further contemplates a composition comprising at least one CXCR4 inhibitor for use in the treatment of cancer or idiopathic pulmonary fibrosis in a subject suffering from said cancer or idiopathic pulmonary fibrosis, wherein the subject is receiving a multi-tyrosine kinase inhibitor therapy. Moreover, encompassed by the present invention is a method for identifying whether a subject suffering from cancer or idiopathic pulmonary fibrosis and receiving a multi-tyrosine kinase inhibitor therapy is susceptible to a CXCR4 inhibitor therapy comprising the steps of determining the CXCR4 signal intensity in a PET dataset obtained from said subject, comparing said CXCR signal intensity to a reference, and identifying whether a subject suffering from cancer or idiopathic pulmonary fibrosis and receiving a multi-tyrosine kinase inhibitor therapy is susceptible for a CXCR4 inhibitor therapy based on the results of the comparison.

### BRIEF DESCRIPTION OF THE DRAWINGS

FIGS. 1A-1D show a CXCR4 PET CT analysis of a patient suffering from idiopathic pulmonary fibrosis prior to Nitedanib therapy (FIG. 1A, FIG. 1B) and after onset of the therapy (FIG. 1C, FIG. 1D). A significant increase in CXCR4 signal is observable.

### DETAILED DESCRIPTION

Idiopathic pulmonary fibrosis as well as metastasizing epithelial cancers such as non-small cell lung carcinoma or breast cancer, usually, have a relatively poor prognosis.

The multi receptor tyrosine kinase inhibitor Nintedanib is known to block signaling by various receptors including the platelet-derived growth factor receptor (PDGFR), fibroblast growth factor receptor (FGFR) and vascular endothelial growth factor receptor (VEGFR). It is believed that Nintedanib reduces disease progression in IPF and slows the decline in lung function by blocking the signaling pathways that are involved in fibrotic processes. Similarly, it has been reported that Nintedanib has an antiangiogenic effect by blocking the VEGFR.

Nintedanib has been approved for the treatment of non-small cell lung carcinoma and idiopathic pulmonary fibrosis in 2015. However, Nintedanib therapy is accompanied by side effects for the patient and its efficacy may be still improved.

CXCR4 is a G-coupled transmembrane receptor of the chemokine family. It is ubiquitously expressed on cells (1). There are several ligands known, however, the pivotal ligand appears to be the stromal cell-derived factor 1 alpha (SDF-1alpha) or CCL12. The receptor and its ligand are both mainly involved in the control of stem cells in the bone marrow and other organs.

In addition to its physiological function, the CXCR4 is typically overexpressed in many tumors of epithelial origin including non-small cell lung carcinoma (NSCLC), breast cancer and pancreatic carcinoma (2, 3). The CXCR4/CCL12 signaling pathways plays an essential role for metastasis but also for proliferation of the tumor. Upon secretion of CCL12, CXCR4 presenting tumor cells are attracted and migrate along the gradient into other tissues. Thus, the local CCL12 production in a certain tissue and the CXCR4 expression rate on tumor cells govern the metastasizing behavior of tumor entity. For several forms of cancer, it could be shown that overexpression of CXCR4 is accompanied by a poor prognosis.

Accordingly, CXCR4 is an interesting target for therapeutics and several clinical trials with CXCR4 are currently underway.

The CXCR4/CCL12 signaling pathway was also shown to be a major player in fibrotic processes. It could be shown hat in patients suffering from idiopathic pulmonary fibrosis the CXCR4 as well as the CCL12 expression is upregulated (4, 5). In different animal models, it could be demonstrated that CXCR4 and CCL12 expression is upregulated and responsible for migration of fibroblast precursor cells from the bone marrow (27). In said models, blocking the signaling pathway resulted in reduced pulmonary fibrosis (4, 6-8). Transforming Growth Factor-beta (TGF-beta) is thought to be the master cytokine of pulmonary fibroses (9). It was recently shown that the signaling pathways of TGF-beta and CXCR4 enhance each other by a potential amplification loop. Said mechanism might be of importance for fibrotic as well as tumor diseases. Moreover, using a tracer, CXCR4 expression could be observed in patients with cancer in vivo by PET analysis (10, 11).

The technical problem underlying the present invention may be seen as the provision of means and methods for complying with the aforementioned needs. The technical problem is solved by the embodiments characterized in the claims and herein below.

The present invention relates to a pharmaceutical composition comprising (i) at least one CXCR4 inhibitor and (ii) a multi-tyrosine kinase inhibitor.

The term “pharmaceutical composition” as used herein refers to a composition for use as a medicament comprising as ingredients the said CXCR4 inhibitor and a multi-tyrosine kinase inhibitor. The said pharmaceutical composition may be used for human or non-human therapy of the diseases referred to herein elsewhere. It will be understood that the pharmaceutical composition comprises or provides the pharmaceutically active ingredients in a therapeutically effective dose. The ingredients, preferably, can be present in liquid or lyophilized form. The pharmaceutical composition is, preferably, for topical or systemic administration. However, depending on the nature and the mode of action of the ingredients, it may be administered by other routes as well. The active ingredients of the composition may be prepared by mixing them and by adding standard pharmaceutical carriers according to conventional procedures. These procedures may involve also mixing, granulating, and compression, or dissolving the ingredients as appropriate to the desired preparation. It will be appreciated that the form and character of the pharmaceutical acceptable carrier or diluent is dictated by the amount of active ingredient with which it is to be combined, the route of administration, and other well-known variables.

A carrier must be acceptable in the sense of being compatible with the other ingredients of the formulation and being not deleterious to the recipient thereof. The pharmaceutical carrier employed may include a solid, a gel, or a liquid. Examples for solid carriers are lactose, terra alba, sucrose, talc, gelatin, agar, pectin, acacia, magnesium stearate, stearic acid and the like. Exemplary of liquid carriers are phosphate buffered saline solution, syrup, oil, water, emulsions, various types of wetting agents, and the like. Similarly, the carrier or diluent may include time delay material well known to the art, such as glyceryl mono-stearate or glyceryl distearate alone or with a wax. Said suitable carriers comprise those mentioned above and others well known in the art and described in standard pharmacopeias.

A diluent is selected so as not to affect the biological activity of the combination. Examples of such diluents are distilled water, physiological saline, Ringer's solutions, dextrose solution, and Hank's solution. In addition, the pharmaceutical composition or formulation may also include other carriers, adjuvants, or non-toxic, non-therapeutic, non-immunogenic stabilizers and the like.

A therapeutically effective dose refers to an amount of the pharmaceutically active ingredients to be used in pharmaceutical composition of the present invention which prevent, ameliorate or treat the symptoms accompanying a disease or condition referred to in this specification. Therapeutic efficacy and toxicity of the ingredients can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. The dosage regimen will be determined by the attending physician and other clinical factors. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment. The pharmaceutical composition referred to herein is administered at least once in order to treat or ameliorate or prevent a disease or condition recited in this specification. However, the said pharmaceutical composition may be administered more than one time. Dosage recommendations shall be indicated in the prescribers or users instructions in order to anticipate dose adjustments depending on the considered recipient.

It is to be understood that the formulation of a medicament takes place under GMP standardized conditions or the like in order to ensure quality, pharmaceutical security, and effectiveness of the medicament.

The term “CXCR4 inhibitor” as used herein refers to one of the active ingredients of the pharmaceutical composition of the present invention. A CXCR4 inhibitor is a compound that is capable of interfering with the signaling activity of the CXCR4 protein such that the said signaling activity is significantly reduced or completely blocked. CXCR4 is a G-coupled transmembrane receptor of the chemokine family It is ubiquitously expressed. There are several ligands known, however, the pivotal ligand appears to be the stromal cell-derived factor 1 alpha (SDF-1alpha) or CCL12. The receptor and its ligand are both mainly involved in the control of stem cells in the bone marrow and other organs. The structure of CXCR4 has been partially resolved and binding sites for antagonistically acting compounds have been identified. Human amino acid sequences for CXCR4 are, preferably, those deposited under GenBank number NP_001008540.1 or NP003458.1. Moreover, CXCR4 as referred to herein encompasses variants of the aforementioned human CXCR4. Such variants have at least the same essential biological and immunological properties human CXCR4. Variants are deemed to share the same essential biological and immunological properties if they are detectable by the same specific assays referred to in this specification, e.g., by FACS—or immunohistochemical assays using polyclonal or monoclonal antibodies specifically recognizing the said human CXCR4. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of human CXCR4. The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman, by the homology alignment algorithm of Needleman and Wunsch, by the search for similarity method of Pearson and Lipman, by computerized implementations of these algorithms GAP, BESTFIT, BLAST, PASTA, and TFASTA in the Wisconsin Genetics Software Package, Genetics Computer Group (GCG), 575 Science Dr., Madison, Wis., or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Variants referred to above may be allelic variants or any other species specific homologs, paralogs, or orthologs. Moreover, the variants referred to herein include fragments of the human CXCR4 or the aforementioned types of variants as long as these fragments have the essential immunological and biological properties as referred to above. Such fragments may be, e.g., degradation products or splice variants of the human CXCR4. Further included are variants which differ due to posttranslational modifications such as phosphorylation or myristylation.

An inhibitor of CXCR4 as referred to herein is, preferably, a compound that binds to CXCR4 and prevents its activation by a natural ligand and, preferably, by CCL12. Accordingly, an inhibitor may bind to the CCL12 binding site thereby preventing CVCL12 binding and subsequent activation of CXCR4. Alternatively, an inhibitor according to the invention may bind at other binding sites outside the CCL12 binding site, whereby, however, the CCL12 binding site becomes sterically blocked or structurally altered upon said binding of the inhibitor (allosteric inhibition) such that binding of the CCL12 ligand and activation of CXCR4 becomes inhibited. Alternatively, the CXCR4 inhibitor in accordance with the present invention may block the signaling inside the cell which is elicited by CCL12 upon binding to CXCR4. For example, an inhibitor may interfere with G-protein activation by CXCR4 upon CCL12 binding.

An inhibitor according to the present invention may reversibly or irreversibly bind to CXCR4. Various compounds have been reported already as CXCR4 inhibitors. These inhibitors belong into different classes of molecules including small molecule inhibitors as well as peptide, protein and, in particular, antibody-based inhibitors. A well known CXCR4 inhibitor is the antagonistically acting compound Plerixafor (AMD3100 or JMD3100) which is commercially available under the trade name Mozobil from Genzyme. Plerixafor is a drug for the treatment of multiple myeloma during HIV therapy. Other CXCR4 antagonists which are currently tested in clinical trials is BL-8040 of Sheba Medical Center and BKT140 from Biokine Therapeutics. Moreover, various blocking antibodies against CXCR4 are under investigation. Bristol Meyers Squibb investigates BMS-936564 an anti-CXCR4 antibody, ALX-0651 is tested by Ablynx. Peptide antagonists such as LY2510924 (Eli Lilly) or MSX-122 (Metastatix) are under investigation in clinical trials as well.

The aforementioned CXCR4 inhibitors can be identified by testing for CXCR4 binding. This can be achieved by contacting the putative CXCR4 inhibitor to CXCR4 and determining binding thereto. Techniques for determining binding are well known in the art and include, e.g., surface plasmon resonance, microscale thermophoresis or dual polarization interferometry. For antibodies as CXCR4, Western blot, Dot blot, affinity chromatography and similar immunological techniques are available as well. Moreover, molecular biological techniques such a phage display or yeast two hybrid assays and others may be also used.

The aforementioned CXCR4 inhibitors can be identified by testing for CXCR4 activity in a cellular test system comprising cells expressing the CXCR4 and CCL12 as activating ligand in the presence and absence of the compound suspected to be a CXCR4 inhibitor, respectively. A CXCR4 inhibitor shall, preferably, inhibit (i.e. prevent or reduce) the signaling elicited by binding of the CCL12 ligand to CXCR4 to a statistically significant extent compared to a control setup. The person skilled in the art is well aware of how such cellular testing systems can be established. Preferably, the test system allows for high-throughput screening such that large libraries of antibodies or small molecule compounds can be efficiently screened for putative CXCR4 inhibitors.

Alternatively, using the structural information available for CXCR4 and the ligand biding site for CCL12, inhibitors may be artificially designed as well.

A small molecule as CXCR4 inhibitor referred to herein may be any type or class of small molecule including naturally occurring small molecules or artificially synthesized small molecules. Typically, a small molecule has a molecular weight lower than 900 Da and, preferably, even lower than 500 Da. A small molecule due to its limited size can diffuse into the cell and act on the Preferably, the small molecule is an organic compound. Besides of artificially generated small molecules, secondary metabolites in bacteria, fungi, plants and animals have turned out to be a good source for biologically active small molecules. Classes of molecules for such secondary metabolite small molecules include alkaloids, glycosides, lipids, non-ribosomal peptides, such as actinomycin-D, phenazines, natural phenols (including flavonoids), polyketide, terpenes, including steroids, and tetrapyrroles.

An antibody as CXCR4 inhibitor referred to herein may be any type or class of antibody including naturally occurring antibodies such as polyclonal antibody sera or monoclonal antibodies. However, an antibody as referred to herein may also be any derivative or variant of such antibodies, preferably, a humanized or chimeric antibody, a single chain antibody, antibody fragments and the like. Antibody fragments and derivatives comprised by the term antibody as used herein encompass a bispecific antibody, a synthetic antibody, an Fab, F(ab)2, Fv, nanobodies or scFv fragment, or a chemically modified derivative of any of these antibodies. Specific binding as used in the context of the antibody of the present invention means that the antibody does not cross react with other polypeptides. Specific binding can be tested by various well known techniques. Antibodies or fragments thereof, in general, can be obtained by using methods described in standard text books of molecular biology. Monoclonal antibodies can be prepared by the techniques which comprise the fusion of mouse myeloma cells to spleen cells derived from immunized mammals and, preferably, immunized mice. Preferably, an immunogenic peptide having the extracellular domain of a CXCR4 is applied to a mammal. The said peptide is, preferably, conjugated to a carrier protein, such as bovine serum albumin, thyroglobulin, and keyhole limpet hemocyanin (KLH). Depending on the host species, various adjuvants can be used to increase the immunological response. Such adjuvants encompass, preferably, Freund's adjuvant, mineral gels, e.g., aluminum hydroxide, and surface active substances, e.g., lysolecithin, pluronic polyols, polyanions, peptides, oil emulsions, keyhole limpet hemocyanin, and dinitrophenol. Monoclonal antibodies which specifically bind to the extracellular domain and, in particular, the ligand binding site of CXCR4 can be subsequently prepared using the well known hybridoma technique, the human B cell hybridoma technique, and the EBV hybridoma technique.

Aptamers as used herein are, preferably, peptide molecules that bind to a specific target molecule. Peptide aptamers are designed to interfere with protein interactions inside cells. They usually comprise of a variable peptide loop attached at both ends to a protein scaffold. This double structural constraint shall increase the binding affinity of the peptide aptamer into the nanomolar range. Said variable peptide loop length is, preferably, composed of ten to twenty amino acids, and the scaffold may be any protein having improved solubility and compacity properties, such as thioredoxin-A. Peptide aptamer selection can be made using different systems including, e.g., the yeast two-hybrid system.

Polypeptides or peptides which bind to the extracellular domain of CXCR4, preferably, encompass peptides and polypeptides which are derived from ligands such as CCL12 or other binding proteins.

Preferably said CXR4 inhibitor is selected from the group consisting of: small molecule CXCR4 antagonists, antibodies, iBodies, peptide antagonists, nanobodies and aptamers.

Yet preferably, the CXCR4 inhibitor may also be an inhibitor of CXCR4 expression. Various nucleic acid inhibitors are known which prevent the expression of given target genes. Preferred nucleic acid inhibitors are ribozymes, antisense nucleic acids, morpholinos, triple-helix forming nucleic acids, siRNAs or micro RNAs. The specificity of these molecules is usually governed by complementary to sequences present in the target, i.e. in the CXCR4 gene or transcript sequence.

Ribozymes are catalytic RNA molecules possessing a well defined tertiary structure that allows for catalyzing either the hydrolysis of one of their own phosphodiester bonds (self-cleaving ribozymes), or the hydrolysis of bonds in other RNAs, but they have also been found to catalyze the aminotransferase activity of the ribosome. The ribozymes envisaged in accordance with the present invention are, preferably, those which specifically hydrolyse the target transcripts. In particular, hammerhead ribozymes are preferred in accordance with the present invention. How to generate and use such ribozymes is well known in the art.

Antisense nucleic acids are, preferably, RNA and comprise a nucleic acid sequence which is essentially or perfectly complementary to the target transcript. Preferably, an antisense nucleic acid essentially consists of a nucleic acid sequence being complementary to at least 100 contiguous nucleotides, more preferably, at least 200, at least 300, at least 400 or at least 500 contiguous nucleotides of the target transcript. How to generate and use antisense nucleic acids is well known in the art.

Morpholinos are synthetic nucleic acid molecules having a length of 20 to 30 nucleotides and, typically 25 nucleotides. Morpholinos bind to complementary sequences of target transcripts by standard nucleic acid base-pairing. They have standard nucleic acid bases which are bound to morpholine rings instead of deoxyribose rings and linked through phosphorodiamidate groups instead of phosphates. Due to replacement of anionic phosphates with the uncharged phosphorodiamidate groups eliminates ionization in the usual physiological pH range, so Morpholinos in organisms or cells are uncharged molecules. The entire backbone of a Morpholino is made from these modified subunits. Unlike inhibitory small RNA molecules, Morpholinos do not degrade their target RNA molecules. Rather, they sterically block binding to a target sequence within a RNA and simply getting in the way of molecules that might otherwise interact with the RNA. How to generate and use such morpholinos is well known in the art.

Triple helix forming nucleic acids are single stranded oligonucleotides which intercalate into genomic DNA and thereby preventing efficient expression. Thus, the target sequences are, typically, within expression control regions such as promoters or translation initiation sites. The triple-helix forming oligonucleotide pairs with the double helix via Hoogsteen hydrogen bonds between the Watson-Crick base pairs. Triple-helix forming oligonucleotides have been developed already successfully as regulators of gene expression. How to generate and use such triple-helix forming nucleic acids is well known in the art.

Small interfering RNAs (siRNAs) are complementary to target RNAs (encoding a gene of interest) and diminish or abolish gene expression by RNA interference (RNAi). Similarly, micro RNAs comprise complementary RNA targeting sequences and also act via RNAi mechanisms. Briefly, the process of RNAi in the cell is initiated by double stranded RNAs (dsRNAs) which are cleaved by a ribonuclease, thus producing siRNA duplexes. The siRNA binds to another intracellular enzyme complex which is thereby activated to target whatever mRNA molecules are homologous (or complementary) to the siRNA sequence. The function of the complex is to target the homologous mRNA molecule through base pairing interactions between one of the siRNA strands and the target mRNA. The mRNA is then cleaved approximately 12 nucleotides from the 3′ terminus of the siRNA and degraded. In this manner, specific mRNAs can be targeted and degraded, thereby resulting in a loss of protein expression from the targeted mRNA. A complementary nucleotide sequence as used herein refers to the region on the RNA strand that is complementary to an RNA transcript of a portion of the target gene. The term “dsRNA” refers to RNA having a duplex structure comprising two complementary and anti-parallel nucleic acid strands. Not all nucleotides of a dsRNA necessarily exhibit complete Watson-Crick base pairs; the two RNA strands may be substantially complementary. The RNA strands forming the dsRNA may have the same or a different number of nucleotides, with the maximum number of base pairs being the number of nucleotides in the shortest strand of the dsRNA. Preferably, the dsRNA is no more than 49, more preferably less than 25, and most preferably between 19 and 23, nucleotides in length. dsRNAs of this length are particularly efficient in inhibiting the expression of the target gene using RNAi techniques. dsRNAs are subsequently degraded by a ribonuclease enzyme into short interfering RNAs (siRNAs). The complementary regions of the siRNA allow sufficient hybridization of the siRNA to the target RNA and thus mediate RNAi. In mammalian cells, siRNAs are approximately 21-25 nucleotides in length. The siRNA sequence needs to be of sufficient length to bring the siRNA and target RNA together through complementary base-pairing interactions. How to generate and use such siRNAs or micro RNAs is well known in the art.

By “at least one CXCR4 inhibitor” it is meant in accordance with the present invention that one or more CXCR4 may be applied. For example two, three, four, five or more different CXCR4 inhibitors may be used in accordance with the present invention. Preferably, the different CXCR4 may be from different classes of inhibitors, e.g., one inhibitor may be a small molecule antagonist, such as, preferably, LY2510924 or BL-8040, whereas the other CXCR4 may be an antibody, such as, preferably, BMS-936564 or MDX-13.

The term “multi-tyrosine kinase inhibitor” as used herein refers to a compound that inhibits at least two different tyrosine kinases and, typically, at least the receptor tyrosine kinases Platelet-derived growth factor receptor (PDGFR), Vascular endothelial growth factor receptor (VEGFR) and Fibroblast growth factor receptor (FGFR). The multiple tyrosine kinase according to the present invention is, thus, not specific for a certain tyrosine kinase but rather may act as a general tyrosine kinase inhibitor or specifically for at least a subset of tyrosine kinases. Tyrosine kinases, in general, act by different mechanisms. They can compete with adenosine triphosphate (ATP), the phosphorylating entity, the substrate or both or can act in an allosteric fashion, namely bind to a site outside the active site, affecting its activity by a conformational change. Moreover, they can deprive tyrosine kinases of access to the Cdc37-Hsp90 molecular chaperone system on which they depend for their cellular stability, leading to their ubiquitylation and degradation. Due to the involvement of improper tyrosine signaling in tumor formation, many tyrosine kinase inhibitors have been approved already as anti tumor drugs. Tyrosine kinase inhibitors affecting two or more tyrosine kinases include Axitinib, Bosutinib, Cabozantinib, Crizotinib, Imatinib, Nilotinib, Nintedanib, Bazopanib, Ponatinib, Sorafenib, Sunitinib or Vandetanib. Preferably, said multi-tyrosine kinase inhibitor is Nintedanib. Nintedanib is an inhibitor for the receptor tyrosine kinases Platelet-derived growth factor receptor (PDGFR), Vascular endothelial growth factor receptor (VEGFR) and Fibroblast growth factor receptor (FGFR) and is an approved drug since 2015 for non-small cell lung carcinoma and idiopathic pulmonary fibrosis.

The pharmaceutical composition according to the present invention is combination of two pharmaceutically active components. Preferably, the said pharmaceutical composition according to the present invention is to be used for the treatment of cancer or idiopathic pulmonary fibrosis in a subject suffering from said cancer or idiopathic pulmonary fibrosis.

Advantageously, it has been suggested in the studies underlying the present invention that side effects of therapies by multi tyrosine kinase inhibitors such as Nintedanib can be prevented or reduced by concomitant administration of a CXCR4 inhibitor. The increased CXCR4 production during multi tyrosine kinase inhibitor therapy, usually, will increase escape mechanism via CXCR4. By blocking the CXCR4 activity, the clinical condition can be significantly improved.

In accordance with the preset invention, thus, compositions comprising both components as active ingredients are provided as well kits comprising them for the concomitant application. Moreover, the invention also provides treatment regimens for the concomitant use of both types of drugs and methods for the treatment of idiopathic pulmonary fibrosis and cancer using the said drugs concomitantly.

Thus, the present invention also relates to a kit comprising (i) at least one CXCR4 inhibitor and (ii) a multi-tyrosine kinase inhibitor.

The term “kit” as used herein refers to a collection comprising at least one CXCR4 inhibitor and a multi-tyrosine kinase inhibitor which provides said ingredients in a manner ready for application. The said kit may comprise additional ingredients or items required for, e.g., administration of the CXCR4 inhibitor and the multi-tyrosine kinase inhibitor. Moreover, the kit may contain instructions for administration including dosage recommendations or dosage regimens. The ingredients of the kit may or may not be package in a single container. Preferably, the kit according to the present invention is to be used for the treatment of cancer or idiopathic pulmonary fibrosis in a subject suffering from said cancer or idiopathic pulmonary fibrosis. The ingredients of the kit may be administered separately to the subject or may be admixed prior to administration.

The present invention also relates to the aforementioned composition or kit for use in the treatment of cancer or idiopathic pulmonary fibrosis in a subject suffering from said cancer or idiopathic pulmonary fibrosis.

Moreover, the present invention also contemplates a composition comprising at least one CXCR4 inhibitor for use in the treatment of cancer or idiopathic pulmonary fibrosis in a subject suffering from said cancer or idiopathic pulmonary fibrosis, wherein the subject is receiving a multi-tyrosine kinase inhibitor therapy.

It will be understood that according to the aforementioned application the at least one CXCR4 inhibitor must not be administered together with the multi tyrosine kinase inhibitor in one composition.

The term “cancer” as used herein refers to any malignant neoplasm resulting from the undesired growth, the invasion, and under certain conditions metastasis of impaired cells in an organism. The cells giving rise to cancer are genetically impaired and have usually lost their ability to control cell division, cell migration behavior, differentiation status and/or cell death machinery. Most cancers form a tumor but some hematopoietic cancers, such as leukemia, do not. Preferably, the cancer according to the present invention is a tumor-forming cancer being capable of metastasizing. More preferably, said cancer is an epithelial cell derived cancer and, most preferably, lung cancer, breast cancer or pancreatic cancer. Among the lung cancer entities, the non-small cell lung carcinoma (NSCLC) is particularly envisaged as cancer in accordance with the present invention. Details of the cancer types may be found in standard text books of medicine.

The term “idiopathic pulmonary fibrosis” as used herein refers to pulmonary fibrosis of unknown cause. Fibrotic processes are, in general, characterized by migration of fibroblasts into other organ tissues. In pulmonary fibrosis, connective tissue cells migrate into the lung and form scar tissue which affects the proper function of the lung (breathing). Idiopathic pulmonary fibrosis can be diagnosed by various imaging techniques including CT and MRT as well as by investigating histologically lung biopsy samples. Further details of the pulmonary fibrosis may be found in standard text books of medicine.

The term “subject” as used herein relates to animals, preferably mammals, and, more preferably, humans. The subject to be treated by a CXCR4 inhibitor in accordance with the present invention shall be a subject receiving a multi-tyrosine kinase inhibitor therapy.

The present invention further contemplates a method for identifying whether a subject suffering from cancer or idiopathic pulmonary fibrosis and receiving a multi-tyrosine kinase inhibitor therapy is susceptible to a CXCR4 inhibitor therapy comprising the steps of:
a) determining the CXCR4 signal intensity in a PET dataset obtained from said subject;
b) comparing said CXCR signal intensity to a reference; and
c) identifying whether a subject suffering from cancer or idiopathic pulmonary fibrosis and receiving a multi-tyrosine kinase inhibitor therapy is susceptible for a CXCR4 inhibitor therapy based on the results of the comparison of step b).

The term “identifying” as used herein means assessing whether a subject will benefit from the treatment with CXCR4 inhibitor in that the medical condition improves or worsening is prevented. As will be understood by those skilled in the art, such an assessment is usually not intended to be correct for all (i.e. 100%) of the subjects to be identified. The term, however, requires that a statistically significant portion of subjects can be identified (e.g. a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc. Details are well known in the art and described in standard text books of statistics. Preferred confidence intervals are at least 90%, at least 95%, at least 97%, at least 98% or at least 99%. The p-values are, preferably, 0.1, 0.05, 0.01, 0.005, or 0.0001. More preferably, at least 60%, at least 70%, at least 80% or at least 90% of the subjects of a population can be properly identified by the method of the present invention.

The term “PET dataset” as used herein refers to an imaging dataset obtained by Positron Emission Tomography (PET) using an affinity nuclear probe. The imaging data contain information in the form of intensity data resembling the amount of nuclear probe bound to its target molecules in the investigated tissue. Thereby, the quantitative amount of target present in a tissue can be visualized in PET scans.

The term “CXCR4 signal intensity” as used herein refers to intensity data resembling the amount of a nuclear probe bound to its target CXCR4 in a tissue. For CXCR4, a specific nuclear probe ⁶⁸Ga-Pentixafor can be preferably be used in PET. Depending on the amount of ⁶⁸Ga-Pentixafor bound to a tissue, the dataset contains intensity data resulting from the ⁶⁸Ga-Pentixafor which represent the amount of CXCR4 present in the tissue. Said intensity data reflect or can be used to calculate the CXCR4 signal intensity, e.g., by quantitatively evaluating the image scan. Techniques for the quantitative evaluation of images are known in the art and include, e.g., the coronal 2-point Dixon 3D volumetric interpolated examination (VIBE) T1w sequence analysis. Preferably, a PET dataset as referred to in accordance with the present invention can be acquired as described in the accompanying Examples below.

The CXCR4 signal intensity in the PET dataset shall subsequently be compared to a reference.

The term “reference” as used herein is the signal intensity for CXCR4 from one or derived from more control datasets or a dataset obtained from subjects or from a dataset obtained from the subject prior to the onset of the multi tyrosine kinase inhibitor therapy.

Preferably, said reference is the CXCR4 signal intensity in a PET dataset obtained from an apparently healthy subject or population thereof or from a subject or group thereof suffering from said idiopathic pulmonary fibrosis or said cancer without receiving multi tyrosine kinase inhibitor therapy. Also preferably, said reference is the CXCR4 signal intensity in a PET dataset obtained from the subject prior to the onset of the multi-tyrosine kinase inhibitor therapy. More preferably, in such a case an increased CXCR4 signal intensity in a PET dataset compared to the reference is indicative for a subject being susceptible to a CXCR4 inhibitor therapy or wherein an essentially identical or decreased CXCR4 signal intensity in a PET dataset compared to the reference is indicative for a subject being not susceptible to a CXCR4 inhibitor therapy.

Alternatively, said reference, preferably, is the CXCR4 signal intensity in a PET dataset obtained from a subject or population thereof known to be susceptible to for a CXCR4 inhibitor therapy. More preferably, in such a case an essentially identical or increased CXCR4 signal intensity in a PET dataset compared to the reference is indicative for a subject being susceptible to a CXCR4 inhibitor therapy or wherein a decreased CXCR4 signal intensity in a PET dataset compared to the reference is indicative for a subject being not susceptible to a CXCR4 inhibitor therapy.

The comparison can be carried out in accordance with the present invention by a data processing device such as a computer which runs an algorithm allowing for a comparison of the signal intensities. It will be understood that the intensity data being the reference and the intensity data derived from the measured dataset shall be of the same category of data, e.g., signal strength or duration, in order to obtain a meaningful result. The skilled person is, however, well aware of how such a comparison can be carried out.

Based on the results of the comparison, the subject suffering from cancer or idiopathic pulmonary fibrosis and receiving a multi-tyrosine kinase inhibitor therapy shall be identified as being susceptible for a CXCR4 inhibitor therapy or not. If the subject is identified as being susceptible for the said therapy, the method may further comprise recommending the said therapy to the patient or the clinician. If the subject is identified as being not susceptible, the method may further comprise recommending no additional CXCR4 therapy for the subject. A “CXCR4 therapy” as referred to herein means a therapy involving administering to the subject at least one CXCR4 inhibitor or a composition of the invention as defined elsewhere herein in detail.

In general, the present invention also provides a method for identifying whether a subject suffering from cancer or idiopathic pulmonary fibrosis and receiving a multi-tyrosine kinase inhibitor therapy is susceptible to a CXCR4 inhibitor therapy comprising the steps of:
a) determining the amount of CXCR4 in a sample of cancer or fibrotic tissue obtained from said subject;
b) comparing said CXCR amount to a reference; and
c) identifying whether a subject suffering from cancer or idiopathic pulmonary fibrosis and receiving a multi-tyrosine kinase inhibitor therapy is susceptible for a CXCR4 inhibitor therapy based on the results of the comparison of step b).

The “sample of cancer or fibrotic tissue” may be, preferably obtained from said subject by biopsy.

The “amount” of CXCR4 in the aforementioned method of the invention may be, preferably, determined by measuring the amount of CXCR4 protein present in the sample. This can be done by immunological methods using antibodies which specifically recognize CXCR4 either in cell lysates obtained from the biopsy sample or on tissue sections. Alternatively, the amount may be determined indirectly by determining the amount of transcripts encoding CXCR4 present in the sample. To this end, nucleic acid detection techniques such as hybridization techniques like Northern Blots or PCR-based techniques may be applied. Typically, these techniques use nucleic acid probes which are capable of specifically hybridizing to the CXCR4 transcripts present in a sample. All detection techniques are well known to those skilled in the art.

The “reference” in this method is an amount of CXCR4 which is present in a sample from a control subject, i.e. a subject suffering from said idiopathic pulmonary fibrosis or said cancer without receiving multi tyrosine kinase inhibitor therapy, or derived from a the amounts of CXCR4 present in samples obtained from a group of such control subjects. Alternatively, the reference may be the amount of CXCR4 found in the subject prior to the onset of the multi tyrosine kinase inhibitor therapy.

Preferably, said reference is the CXCR4 amount in a sample obtained from an apparently healthy subject or population thereof or from a subject or group thereof suffering from said idiopathic pulmonary fibrosis or said cancer without receiving multi tyrosine kinase inhibitor therapy. Also preferably, said reference is the CXCR4 amount in a sample obtained from the subject prior to the onset of the multi-tyrosine kinase inhibitor therapy. More preferably, in such a case an increased CXCR4 amount compared to the reference is indicative for a subject being susceptible to a CXCR4 inhibitor therapy or wherein an essentially identical or decreased CXCR4 amount compared to the reference is indicative for a subject being not susceptible to a CXCR4 inhibitor therapy.

Alternatively, said reference, preferably, is the CXCR4 amount in a sample obtained from a subject or population thereof known to be susceptible to for a CXCR4 inhibitor therapy. More preferably, in such a case an essentially identical or increased CXCR4 amount compared to the reference is indicative for a subject being susceptible to a CXCR4 inhibitor therapy or wherein a decreased CXCR4 amount compared to the reference is indicative for a subject being not susceptible to a CXCR4 inhibitor therapy.

The comparison can be carried out in accordance with the present invention by a data processing device such as a computer which runs an algorithm allowing for a comparison of the values for the amounts. The skilled person is, however, well aware of how such a comparison can be carried out.

Based on the results of the comparison, the subject suffering from cancer or idiopathic pulmonary fibrosis and receiving a multi-tyrosine kinase inhibitor therapy shall be identified as being susceptible for a CXCR4 inhibitor therapy or not. If the subject is identified as being susceptible for the said therapy, the method may further comprise recommending the said therapy to the patient or the clinician. If the subject is identified as being not susceptible, the method may further comprise recommending no additional CXCR4 therapy for the subject.

The present invention also provides for a kit for carrying out the aforementioned methods of the invention for method for identifying whether a subject suffering from cancer or idiopathic pulmonary fibrosis and receiving a multi-tyrosine kinase inhibitor therapy is susceptible to a CXCR4 inhibitor therapy. The said kit shall comprise either the specific tracer ⁶⁸Ga-Pentixafor or a detection agent, such as an antibody or a nucleic acid probe, which specifically recognizes the CXCR4 protein and, thus, allows determining its amount in a biopsy sample. Preferably, the kit comprises theses agents in a ready-to-use formulation. Moreover, the kit, preferably, also comprises instructions for carrying out the said methods and, preferably, also a computer program code on a storage device for carrying out the comparison.

In the following, particular embodiments of the present invention are summarized. However, these embodiments shall not be construed to limit the invention.
1. A pharmaceutical composition comprising: (i) at least one CXCR4 inhibitor and (ii) a multi-tyrosine kinase inhibitor.
2. A kit comprising: (i) at least one CXCR4 inhibitor and (ii) a multi-tyrosine kinase inhibitor.
3. The composition or kit of embodiment 1 or 2, wherein said CXCR4 inhibitor is selected from the group consisting of: small molecule CXCR4 antagonists and, preferably, LY2510934 or BL-8040, antibodies and, preferably, BMS-93654 or MDX-13, iBodies, peptid antagonists, nanobodies and aptamers or is selected from the group consisting of: ribozymes, antisense nucleic acids, morpholinos, triple-helix forming nucleic acids, siRNAs and micro RNAs.
4. The composition or kit of any one of embodiments 1 to 3, wherein said multi-tyrosine kinase inhibitor is Nintedanib.
5. A composition or kit of any one of embodiments 1 to 4 for use in the treatment of cancer or idiopathic pulmonary fibrosis in a subject suffering from said cancer or idiopathic pulmonary fibrosis.
6. A composition comprising at least one CXCR4 inhibitor for use in the treatment of cancer or idiopathic pulmonary fibrosis in a subject suffering from said cancer or idiopathic pulmonary fibrosis, wherein the subject is receiving a multi-tyrosine kinase inhibitor therapy.
7. The composition for use of embodiment 6, wherein said CXCR4 inhibitor is selected from the group consisting of: small molecule CXCR4 antagonists and, preferably, LY2510934 or BL-8040, antibodies and, preferably, BMS-93654 or MDX-13, iBodies, peptid antagonists, nanobodies and aptamers or is selected from the group consisting of: ribozymes, antisense nucleic acids, morpholinos, triple-helix forming nucleic acids, siRNAs and micro RNAs.
8. The composition for use of embodiment 6 or 7, wherein said multi-tyrosine kinase inhibitor is Nintedanib.
9. The composition for use of any one of embodiments 6 to 8, wherein said cancer is lung cancer, breast cancer or pancreatic cancer.
10. A method for identifying whether a subject suffering from cancer or idiopathic pulmonary fibrosis and receiving a multi-tyrosine kinase inhibitor therapy is susceptible to a CXCR4 inhibitor therapy comprising the steps of:
   a) determining the CXCR4 signal intensity in a PET dataset obtained from said subject;
   b) comparing said CXCR signal intensity to a reference; and
   c) identifying whether a subject suffering from cancer or idiopathic pulmonary fibrosis and receiving a multi-tyrosine kinase inhibitor therapy is susceptible for a CXCR4 inhibitor therapy based on the results of the comparison of step b).
11. The method of embodiment 10, wherein said CXR4 inhibitor is selected from the group consisting of: small molecule CXCR4 antagonists and, preferably, LY2510934 or BL-8040, antibodies and, preferably, BMS-93654 or MDX-13, iBodies, peptid antagonists, nanobodies and aptamers or is selected from the group consisting of: ribozymes, antisense nucleic acids, morpholinos, triple-helix forming nucleic acids, siRNAs and micro RNAs.
12. The method of embodiment 10 or 11, wherein said multi-tyrosine kinase inhibitor is Nintedanib.
13. The method of any one of embodiments 10 to 12, wherein said reference is the CXCR4 signal intensity in a PET dataset obtained from an apparently healthy subject or population thereof or from a subject or group thereof suffering from said idiopathic pulmonary fibrosis or said cancer without receiving multi tyrosine kinase inhibitor therapy.
14. The method of any one of embodiments 10 to 13, wherein said reference is the CXCR4 signal intensity in a PET dataset obtained from the subject prior to the onset of the multi-tyrosine kinase inhibitor therapy.
15. The method of embodiment 13 or 14, wherein an increased CXCR4 signal intensity in a PET dataset compared to the reference is indicative for a subject being susceptible to a CXCR4 inhibitor therapy or wherein an essentially identical or decreased CXCR4 signal intensity in a PET dataset compared to the reference is indicative for a subject being not susceptible to a CXCR4 inhibitor therapy.
16. The method of any one of embodiments 10 to 12, wherein said reference is the CXCR4 signal intensity in a PET dataset obtained from a subject or population thereof known to be susceptible to for a CXCR4 inhibitor therapy.
17. The method of embodiment 16, wherein an essentially identical or increased CXCR4 signal intensity in a PET dataset compared to the reference is indicative for a subject being susceptible to a CXCR4 inhibitor therapy or wherein a decreased CXCR4 signal intensity in a PET dataset compared to the reference is indicative for a subject being not susceptible to a CXCR4 inhibitor therapy.
18. A method for identifying whether a subject suffering from cancer or idiopathic pulmonary fibrosis and receiving a multi-tyrosine kinase inhibitor therapy is susceptible to a CXCR4 inhibitor therapy comprising the steps of:
   a) determining the amount of CXCR4 in a sample of cancer or fibrotic tissue obtained from said subject;
   b) comparing said CXCR amount to a reference; and
   c) identifying whether a subject suffering from cancer or idiopathic pulmonary fibrosis and receiving a multi-tyrosine kinase inhibitor therapy is susceptible for a CXCR4 inhibitor therapy based on the results of the comparison of step b).
19. The method of embodiment 10, wherein said CXR4 inhibitor is selected from the group consisting of: small molecule CXCR4 antagonists and, preferably, LY2510934 or BL-8040, antibodies and, preferably, BMS-93654 or MDX-13, iBodies, peptid antagonists, nanobodies and aptamers or is selected from the group consisting of: ribozymes, antisense nucleic acids, morpholinos, triple-helix forming nucleic acids, siRNAs and micro RNAs.
20. The method of embodiment 18 or 19, wherein said multi-tyrosine kinase inhibitor is Nintedanib.
21. The method of any one of embodiments 18 to 20, wherein said reference is the CXCR4 signal intensity in a PET dataset obtained from an apparently healthy subject or population thereof or from a subject or group thereof suffering from said idiopathic pulmonary fibrosis or said cancer without receiving multi tyrosine kinase inhibitor therapy.
22. The method of any one of embodiments 18 to 21, wherein said reference is the CXCR4 signal intensity in a PET dataset obtained from the subject prior to the onset of the multi-tyrosine kinase inhibitor therapy.
23. The method of embodiment 21 or 22, wherein an increased CXCR4 signal intensity in a PET dataset compared to the reference is indicative for a subject being susceptible to a CXCR4 inhibitor therapy or wherein an essentially identical or decreased CXCR4 signal intensity in a PET dataset compared to the reference is indicative for a subject being not susceptible to a CXCR4 inhibitor therapy.
24. The method of any one of embodiments 18 to 20, wherein said reference is the CXCR4 signal intensity in a PET dataset obtained from a subject or population thereof known to be susceptible to for a CXCR4 inhibitor therapy.
25. The method of embodiment 24, wherein an essentially identical or increased CXCR4 signal intensity in a PET dataset compared to the reference is indicative for a subject being susceptible to a CXCR4 inhibitor therapy or wherein a decreased CXCR4 signal intensity in a PET dataset compared to the reference is indicative for a subject being not susceptible to a CXCR4 inhibitor therapy.

All references cited in this specification are herewith incorporated by reference with respect to their entire disclosure content and the disclosure content specifically mentioned in this specification.

### EXAMPLES

### Example 1: CXCR4 Abundance in Patients with Idiopathic Pulmonary Fibrosis Prior and After the Onset of Nintedanib Therapy

Patients were diagnosed with clinical idiopathic pulmonary fibrosis according to diagnostic criteria of the American Thoracic Society/European Respiratory Society.

Pentixafor synthesis. Material for synthesis of ⁶⁸Ga-pentixafor (CPCR4.2) was provided by Scintomics (Fürstenfeldbruck, Germany) Synthesis was performed as previously described (18, 19), using a ⁶⁸Ge/⁶⁸Ga-generator (Eckert&Ziegler, Braunschweig, Germany) connected to a Scintomics GRP synthesis module.

Clinical Imaging. All studies were obtained on a dedicated PET/CT system (Biograph mCT 128 Flow; Siemens, Knoxville, USA), equipped with an extended field-of-view LSO PET component, a 128-slice spiral CT component, and a magnetically driven table optimized for continuous scanning The patients received an intravenous injection of 110 (IQR, 80-120) MBq of ⁶⁸Ga-pentixafor. Imaging started with a low-dose non-enhanced helical CT (120 kV, mA modulated, pitch 1.2, reconstructed axial slice thickness 5.0 mm) performed for attenuation correction of PET acquisitions. PET images of the whole body were then acquired using continuous bed motion (CBM) at a speed of 2.0 mm/s for head and neck and 0.5 mm/s for chest and abdomen at 60 min post injection. All studies were reconstructed using time-of-flight and point-spread function information combined with an iterative algorithm (Ultra HD®, Siemens Healthcare; 2 iterations, 21 subsets, matrix 200; zoom 1.0; Gaussian filter of 5.0). In addition, a non-contrast breath-hold high-resolution CT (HRCT) (120 kV, mA modulated) of the chest was used to obtain contiguous 1.0-mm cross-sectional slices throughout the thorax. Raw data were reconstructed using the reconstruction kernels B31f and B70f and displayed at window settings suitable for viewing the lung parenchyma.

PET/CT image analysis. Transaxial PET, CT and fused PET/CT 68Ga-pentixafor PET/CT images were analyzed on a dedicated workstation equipped with a commercial software package (syngo.via; Siemens Healthcare).

PET images were visually evaluated for the presence of elevated radiotracer uptake in lung parenchyma in areas of usual interstitial pneumonia (UIP) pattern on CT. Then, ⁶⁸Ga-Pentixafor uptake was quantified using a 3D Volume-of-Interest (VOI) technique with isocontour thresholding, yielding mean and peak standardized uptake values (SUVₘₑₐₙ and SUVₚₑₐₖ). Separate measurements were performed in three subpleural regions in each lung lobe, and values for each region were then averaged for the subsequent statistical analysis. Tracer uptake in mediastinal and hilar lymph nodes was assessed in three thoracic lymph node stations using 3D VOIs with isocontour thresholding, and also averaged for the subsequent statistical analysis. In addition, tracer uptake (SUVₘₑₐₙ) in spleen and bone marrow was mean, recorded to evaluate systemic interactions.

Patients suffering from idiopathic pulmonary fibrosis were investigated for CXCR4 abundance by PET/CT using a CXCR4 tracer ⁶⁸Ga-Pentixafor as described.

The mean signal intensities (SUVₘₑₐₙ) were determined in different regions, i.e. upper-, middle and lower fields. The volume of CXCR4-positive areas were determined and put into relation to the total volume of the respective areas. The extent of the fibrotic areas were judged in PET/CT and the SUVₘₑₐₙ was determined for mediastinal lymph nodes, spleen and bone marrow.

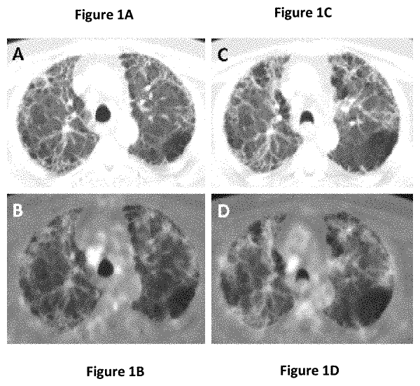

Upon treatment of the patients with Nintedanib, an increase of CXCR4 could be observed (FIG. 1).

3 out of 5 analyzed patients showed a strong increase in CXCR4 signal intensity under Nintedanib treatment in the fibrotic areas in the lung but not in lymph nodes. The said 3 patients received Nintedanib in a dosage of 150 mg at the day of the PET/CT investigation in the morning hours. On patient showed a constant CXCR4 signal, while one patient suffering from familial idiopathic pulmonary fibrosis showed a decrease in signal intensity. In the latter case, it is unclear whether the patient had received the Nintedanib treatment on the day of the investigation at all.

PET/CT was also used in a cohort study of 12 patients suffering from idiopathic pulmonary fibrosis and receiving Pirfenidone treatment for determining the CXCR4 signal intensity. Pirfenidon is a drug which is also used fort he treatment of idiopathic pulmonary fibrosis. However, contrary to the observations made for Nintedanib therapy, the signal intensities for CXCR4 remained constant or even decreased in said patients.

The differences between the CXCR4 signal intensities between baseline and “under Pirfenidone therapy” correlate with the clinical development (pulmonary function, forced vital capacity (FVC)) A strong increase in pulmonary fibrotic areas as observed for Nintedanib, was not observed in patients treated with Pirfenidone.

Based on the present observations, it is reasonable to speculate that the increase in CXCR4 levels under Nintedanib treatment in patients with idiopathic pulmonary fibrosis or cancer is part of an escape mechanism. Thus, the administration of Nintedanib together with a CXCR4 inhibitor blocking said signaling pathway shall be a promising therapeutic concept avoiding escapers. Several CXCR4 inhibitors are available and currently under clinical investigation. Theses inhibitors include, e.g., Plerixafor (AMD3100 or JMD3100, Trade name Mozobil; Genzyme, US), the blocking antibody BMS 936564 (Bristol Myers Squibb, US), BKT140 (Biokine Therapeutics, US), the nanobody ALX-0651 (Ablynx, US), the peptide antagonist LY2510924 (Durvalumab; Eli Lilly, US), MSX-122 (Metastatix, US), BL-8040 (Sheba Medical Center). Moreover, the CXCR4 PET/CT can be used as a biomarker for assessing the usefulness of such a combined therapy.

### CITED REFERENCES

1. Burger J A, Kipps T J. CXCR4: a key receptor in the crosstalk between tumor cells and their microenvironment. Blood 2006; 107:1761-7.
2. Zhao H, Guo L, Zhao H, Zhao J, Weng H, Zhao B. CXCR4 over-expression and survival in cancer: a system review and meta-analysis. Oncotarget 2015; 6:5022-40.
3. Phillips R J, Burdick M D, Lutz M, Belperio J A, Keane M P, Stricter R M. The stromal derived factor-1/CXCL12-CXC chemokine receptor 4 biological axis in non-small cell lung cancer metastases. Am J Respir Crit Care Med 2003; 167:1676-86.
4. Xu J, Mora A, Shim H, Stecenko A, Brigham K L, Rojas M. Role of the SDF-1/CXCR4 axis in the pathogenesis of lung injury and fibrosis. Am J Respir Cell Mol Biol 2007; 37:291-9.
5. Antoniou K M, Soufla G, Lymbouridou R, et al. Expression analysis of angiogenic growth factors and biological axis CXCL12/CXCR4 axis in idiopathic pulmonary fibrosis. Connect Tissue Res 2010; 51:71-80.
6. Makino H, Aono Y, Azuma M, et al. Antifibrotic effects of CXCR4 antagonist in bleomycin-induced pulmonary fibrosis in mice. J Med Invest 2013; 60:127-37.
7. Song J S, Kang C M, Kang H H, et al. Inhibitory effect of CXC chemokine receptor 4 antagonist AMD3100 on bleomycin induced murine pulmonary fibrosis. Exp Mol Med 2010; 42:465-72.
8. Shu H K, Yoon Y, Hong S, et al. Inhibition of the CXCL12/CXCR4-axis as preventive therapy for radiation-induced pulmonary fibrosis. PLoS One 2013; 8:e79768.
9. King T E, Jr., Pardo A, Selman M. Idiopathic pulmonary fibrosis. Lancet 2011; 378:1949-61.
10. Vag T, Gerngross C, Herhaus P, et al. First Experience on Chemokine Receptor CXCR4 Targeted Positron Emission Tomography (PET) Imaging in Patients with Solid Cancers. J Nucl Med 2016.
11. Derlin T, Jonigk D, Bauersachs J, Bengel F M. Molecular Imaging of Chemokine Receptor CXCR4 in Non-Small Cell Lung Cancer Using 68Ga-Pentixafor PET/CT: Comparison With 18F-FDG. Clin Nucl Med 2016.

1.-15. (canceled)

16. A pharmaceutical composition comprising: (i) at least one CXCR4 inhibitor and (ii) a multi-tyrosine kinase inhibitor.

17. The pharmaceutical composition of claim 16, wherein said CXR4 inhibitor is selected from the group consisting of: small molecule CXCR4 antagonists and, preferably, LY2510934 or BL-8040, antibodies and, preferably, BMS-93654 or MDX-13, iBodies, peptid antagonists, nanobodies and aptamers or is selected from the group consisting of: ribozymes, antisense nucleic acids, morpholinos, triple-helix forming nucleic acids, siRNAs and micro RNAs.

18. The pharmaceutical composition of claim 16, wherein said multi-tyrosine kinase inhibitor is Nintedanib.

19. A method for treating cancer or idiopathic pulmonary fibrosis in a subject suffering from said cancer or said idiopathic pulmonary fibrosis comprising contacting said subject to (i) at least one CXCR4 inhibitor and (ii) a multi-tyrosine kinase inhibitor, thereby treating said cancer or said idiopathic pulmonary fibrosis.

20. The method of claim 19, wherein the subject is receiving a multi-tyrosine kinase inhibitor therapy.

21. The method of claim 20, wherein said CXCR4 inhibitor is selected from the group consisting of: small molecule CXCR4 antagonists and, preferably, LY2510934 or BL-8040, antibodies and, preferably, BMS-93654 or MDX-13, iBodies, peptid antagonists, nanobodies and aptamers or is selected from the group consisting of: ribozymes, antisense nucleic acids, morpholinos, triple-helix forming nucleic acids, siRNAs and micro RNAs.

22. The method of claim 20, wherein said multi-tyrosine kinase inhibitor is Nintedanib.

23. The method of claim 20, wherein said cancer is lung cancer, breast cancer or pancreatic cancer.

24. A method for identifying whether a subject suffering from cancer or idiopathic pulmonary fibrosis and receiving a multi-tyrosine kinase inhibitor therapy is susceptible to a CXCR4 inhibitor therapy comprising the steps of:
a) determining the CXCR4 signal intensity in a PET dataset obtained from said subject;
b) comparing said CXCR signal intensity to a reference; and
c) identifying whether a subject suffering from cancer or idiopathic pulmonary fibrosis and receiving a multi-tyrosine kinase inhibitor therapy is susceptible for a CXCR4 inhibitor therapy based on the results of the comparison of step b).

25. The method of claim 24, wherein said CXR4 inhibitor is selected from the group consisting of: small molecule CXCR4 antagonists and, preferably, LY2510934 or BL-8040, antibodies and, preferably, BMS-93654 or MDX-13, iBodies, peptid antagonists, nanobodies and aptamers or is selected from the group consisting of: ribozymes, antisense nucleic acids, morpholinos, triple-helix forming nucleic acids, siRNAs and micro RNAs.

26. The method of claim 24, wherein said multi-tyrosine kinase inhibitor is Nintedanib.

27. The method of claim 24, wherein said reference is the CXCR4 signal intensity in a PET dataset obtained from an apparently healthy subject or population thereof or from a subject or group thereof suffering from said idiopathic pulmonary fibrosis or said cancer without receiving multi tyrosine kinase inhibitor therapy.

28. The method of claim 24, wherein said reference is the CXCR4 signal intensity in a PET dataset obtained from the subject prior to the onset of the multi-tyrosine kinase inhibitor therapy.

29. The method of claim 27, wherein an increased CXCR4 signal intensity in a PET dataset compared to the reference is indicative for a subject being susceptible to a CXCR4 inhibitor therapy or wherein an essentially identical or decreased CXCR4 signal intensity in a PET dataset compared to the reference is indicative for a subject being not susceptible to a CXCR4 inhibitor therapy.

30. The method of claim 24, wherein said reference is the CXCR4 signal intensity in a PET dataset obtained from a subject or population thereof known to be susceptible to for a CXCR4 inhibitor therapy.

31. The method of claim 30, wherein an essentially identical or increased CXCR4 signal intensity in a PET dataset compared to the reference is indicative for a subject being susceptible to a CXCR4 inhibitor therapy or wherein a decreased CXCR4 signal intensity in a PET dataset compared to the reference is indicative for a subject being not susceptible to a CXCR4 inhibitor therapy.

32. A kit comprising: (i) at least one CXCR4 inhibitor and (ii) a multi-tyrosine kinase inhibitor.
